(19) United States
(12) Patent Application Publication
(10) Pub. No.: US 2016/0151209 A1
(43) Pub. Date: Jun. 2, 2016

(54) Orthopaedic Cast Tacks

(71) Applicant: Paul Douglas PATERSON, West Falls, NY (US)

(72) Inventor: Paul D Paterson, West Falls, NY (US)

(21) Appl. No.: 14/905,061

(22) PCT Filed: Jul. 15, 2013
(86) PCT No.: PCT/US2013/050439

§ 371 Date: Jan. 14, 2016

(51) Int. Cl. A61F 13/04; A61F 13/10

(52) U.S. Cl. CPC A61F 13/04; A61F 13/107

(57) ABSTRACT

The present disclosure relates to adorned orthopaedic casts, components thereof, and systems and methods for making and using such casts and components. Adorned cast components provided herein include fitments and accoutrements, where a fitment is partially embedded in a casting composition on a body surface such that an accoutrement can reversibly engage the fitment.

### CROSS-REFERENCE TO RELATED APPLICATIONS

This application is a 35 U.S.C. 371 National Stage entry of PCT/US2013/050439, filed Jul. 15, 2013, the entire contents of which is hereby incorporated by reference in its entirety.

### FIELD OF THE INVENTION

The present disclosure relates generally to casts and features thereof. In particular, the present invention encompasses adorned orthopaedic casts, components of adorned orthopaedic casts, and systems and methods for making and using such casts to facilitate patient compliance via aesthetic enhancement.

### BACKGROUND OF THE INVENTION

The following description is provided to assist the understanding of the reader. None of the information provided or references cited is admitted to be prior art.

Orthopaedic intervention is necessary for the treatment of physical injuries to the bone, e.g., fractured or broken bones, which are a common occurrence worldwide. Indeed, roughly seven million of such injuries require medical attention in the United States on an annual basis. Together with various demographic considerations, e.g., age, weight and gender, common indications for bone injuries include an individual's bone fracture score, i.e., prognostic determinations based on algorithmic input factors, and their respective pathological status such as, for example, the presence or absence of tumors and osteoporosis, among other diseases or conditions. In developed countries, moreover, an individual can expect to suffer two bone fractures on average during their lifetime. Orthopaedic casts to this end are necessary for fracture-site immobilization, proper alignment and osteoinductive recovery.

Complete recovery, however, can take twelve weeks or longer. In this respect, although a wide range of casting applications and materials impart a variety of options for injury site immobilization, such materials nevertheless remain on the body throughout the healing process. In addition to the discomfort and inconvenience accompanying extended therapeutic immobilization, the prospect of having a limb encased by a large, conspicuous bandage composed of plaster or fiberglass is an aesthetically undesirable disposition for many patients, including injured children. New cast applications are therefore needed to ensure fracture-site healing while functioning in concert with an individual's aesthetic preferences. Such therapeutic indications possess the greatest benefit as minimally disruptive enhancements for improving compliance and aesthetic appeal.

### SUMMARY OF THE INVENTION

In one aspect, the present invention provides an adorned cast that includes a casting composition entailing at least one layer of reticulated material configured to conform to a body surface that defines a casting region, where the casting region is substantially enveloped by the casting composition to form an orthopaedic cast; one or more fitments at least partially embedded in the orthopaedic cast, where the one or more fitments comprise a buttress end, engagement post, and an apex; and one or more accoutrements capable of cooperatively interacting with the one or more fitments. In some embodiments, an engagement post shell is included. The engagement post shell reversibly envelopes the apex and the engagement post, and the engagement post shell reversibly contacts an upper surface of the buttress end in respective embodiments.

In illustrative embodiments, the reticulated material is selected from silicone, rubber, vinyl, non-water hardenable urethane, plastic, plastic-based materials, fiberglass, metal, ceramic, one or more polymers, resin, plaster, scented plastic, cellulose, and scented cellulose, or any combination thereof. In suitable embodiments, the body surface is selected from an entire body, lower extremity limbs, upper extremity limbs, one or both shoulders, one or both hips, one or both arms, one or both elbows, one or both hands, one or both wrists, one or both legs, one or both feet, one or both ankles, one or both heels, one or both knees, neck, and head, or any combination thereof. In some embodiments, the casting region allows for application of the cast to an entire body, lower extremity limbs, upper extremity limbs, one or both shoulders, one or both hips, one or both arms, one or both elbows, one or both hands, one or both wrists, one or both legs, one or both feet, one or both ankles, one or both heels, one or both knees, neck, and head, or any combination thereof.

The orthopaedic cast is selected from a spica cast and a thoracolumbar cast in suitable embodiments. In certain embodiments, the one or more fitments are positioned within and/or throughout the orthopaedic cast in a manner selected from angled, straight, slanted, tapered, orthogonal, parallel, perpendicular, curved, diagonal, random, or any combination thereof. In some embodiments, the height of the one or more fitments is from about 0.01 cm to about 10 cm. In illustrative embodiments, the one or more fitments are composed of silicone, rubber, vinyl, non-water hardenable urethane, plastic, plastic-based materials, fiberglass, metal, ceramic, one or more polymers, resin, plaster, scented plastic, cellulose, and scented cellulose, or any combination thereof.

In various embodiments, the buttress end, engagement post, and apex of the one or more fitments are integrated to form a single structure. The buttress end, engagement post, and apex of the one or more fitments are, in some embodiments, reversibly connected to form a single structure. In illustrative embodiments, the buttress end, engagement post, and apex of the one or more fitments are separate structures capable of reversibly connecting to form a single structure. In suitable embodiments, the buttress end comprises an upper surface and a lower surface. In illustrative embodiments, the upper surface of the buttress end includes one or more adherence nodes to stabilize the one or more fitments. The lower surface of the buttress end is convex in certain embodiments. In illustrative embodiments, the lower surface of the buttress end possesses one or more adherence nodes, while partially contacting the casting region. Likewise, in suitable embodiments, the upper surface of the buttress end contacts an interior region of the orthopaedic cast.

In illustrative embodiments, the engagement post connects the buttress end and the apex. The engagement post is partially embedded in the casting material and/or the orthopaedic cast in certain embodiments. In illustrative embodiments, the apex extends beyond the plane of an exterior surface of the orthopaedic cast, while illustrative embodiments provide for an apex shape selected from circular, rectangular, coned, pyramidal, grooved, square, polygonal, curved, concentric, concave, perimetric, diamond, hexagonal, or triangular configurations, or any combination thereof. In some embodiments, the apex is configured to secure the one or more fitments in place. To this end, the securing is selected from snapping, screwing, clamping, adhering, locking, riveting, frictional fitting, bayonetting, or any combination thereof in suitable embodiments.

In certain embodiments, the engagement post shell reversibly envelopes the apex and the engagement post. Likewise, the engagement post shell reversibly contacts an upper surface of the buttress end in some embodiments. The engagement post, moreover, connects the buttress end and the apex in certain embodiments, while the engagement post is solid or has a hollow interior region in illustrative embodiments. In suitable embodiments, the apex extends beyond the plane of an exterior surface of the orthopaedic cast, while, in other embodiments, the apex is excised leaving the one or more fitments flush with the exterior surface of the orthopaedic cast. The one or more accoutrements cooperatively interact with the apex of the one or more fitments when the one or more accoutrements are substantially aligned with the apex, i.e., when the apex is not excised. In other embodiments, the one or more accoutrements further possess an engagement rod, where the engagement rod cooperatively interacts with the one or more fitments when the apex has been excised. The cooperative interaction is between the engagement rod and the hollow interior region of the engagement post in particular embodiments.

In certain embodiments, the one or more accoutrements cooperatively interact with the apex of the one or more fitments when the one or more accoutrements are substantially aligned with the apex. The cooperative interaction is selected from snapping, screwing, clamping, adhering, locking, riveting, frictional fitting, bayonetting, or any combination thereof in certain embodiments. In illustrative embodiments, the one or more accoutrements are composed of silicone, rubber, vinyl, non-water hardenable urethane, plastic, plastic-based materials, fiberglass, metal, ceramic, polymers, resin, plaster, scented plastic, cellulose, or scented cellulose, or any combination thereof.

In some embodiments, the one or more accoutrements have a shape selected from polygonal, rectangular, square, circular, curved, concentric, concave, perimetric, diamond, hexagonal, or triangular configurations, or any combination thereof. In illustrative embodiments, the one or more accoutrements are removable or interchangeable, or both. The one or more accoutrements have a surface, and wherein the surface is decorated, colored, patterned, texturized, designable, colorimetric, modifiable, or designable, or any combination thereof in some embodiments.

In one aspect, the present invention provides a system for adorning an orthopaedic cast, including a casting composition to form the orthopaedic cast, one or more fitments having a buttress end, an engagement post, and an apex, where the engagement post connects the buttress end to the apex such that when force is applied to the buttress end the apex is impelled through the casting material, and one or more accoutrements that cooperatively interact with the apex to form an adorned orthopaedic cast. In illustrative embodiments, the system further includes an engagement post shell to facilitate application of the one or more fitments. In illustrative embodiments, the engagement post shell reversibly envelopes the apex and the engagement post, and where the engagement post shell reversibly contacts an upper surface of the buttress end. In some embodiments, the engagement post shell reversibly contacts an upper surface of the buttress end.

In suitable embodiments, the engagement post connects the buttress end and the apex, where the engagement post is solid or possesses a hollow interior region. In some embodiments, the apex extends beyond the plane of an exterior surface of the orthopaedic cast, while in other embodiments the apex is excised. In particular embodiments, the one or more accoutrements cooperatively interact with the apex of the one or more fitments when the one or more accoutrements are substantially aligned with the apex. In other embodiments, the one or more accoutrements cooperatively interact with the engagement post of the one or more fitments when the one or more accoutrements possess an engagement rod.

In illustrative embodiments, the present systems include a casting composition is selected from silicone, rubber, vinyl, non-water hardenable urethane, plastic, plastic-based materials, fiberglass, metal, ceramic, one or more polymers, resin, plaster, scented plastic, cellulose, and scented cellulose, or any combination thereof. In suitable embodiments, the height of the one or more fitments is from about 0.01 cm to about 10 cm. In illustrative embodiments, the one or more fitments are composed of silicone, rubber, vinyl, non-water hardenable urethane, plastic, plastic-based materials, fiberglass, metal, ceramic, one or more polymers, resin, plaster, scented plastic, cellulose, and scented cellulose, or any combination thereof. In certain embodiments, the buttress end, engagement post, and apex of the one or more fitments are integrated to form a single structure.

In suitable systematic embodiments, the buttress end, engagement post, and apex of the one or more fitments are reversibly connected to form a single structure. The buttress end, engagement post, and apex of the one or more fitments, moreover, are separate structures in some embodiments that are capable of reversibly connecting to form a single structure. In illustrative embodiments, the single structure functions to perforate the casting material, secure the one or more fitments in place or operate as a docking component for the one or more accoutrements. In illustrative embodiments, the buttress end has an upper surface and a lower surface. In some embodiments, the upper surface of the buttress end is planar.

In some embodiments, the upper surface of the buttress end contacts an interior region of the orthopaedic cast. In illustrative embodiments, the lower surface of the buttress end is convex. The lower surface of the buttress end, in certain embodiments, possesses one or more adherence nodes, while partially contacting the casting region. In some embodiments, pressure and/or force is applied to the buttress end, specifically the lower surface of the buttress end. In illustrative embodiments, the pressure and/or force applied to the buttress end is sufficient to perforate the apex through the casting material and at least partially embed the engagement post within the casting material.

In suitable embodiments, the engagement post connects the buttress end and the apex. And, in some embodiments, the engagement post is at least partially embedded in the casting material and/or the orthopaedic cast. In illustrative embodiments, the apex extends beyond the plane of an exterior surface of the orthopaedic cast. In suitable embodiments, the apex has a shape selected from circular, rectangular, coned, pyramidal, grooved, square, polygonal, curved, concentric, concave, perimetric, diamond, hexagonal, or triangular configurations, or any combination thereof. In illustrative embodiments, the apex is configured to secure the one or more fitments in place. In some embodiments, the securing is selected from the group consisting of snapping, screwing, clamping, adhering, locking, riveting, frictional fitting, bayonetting, or any combination thereof. In suitable embodiments, the one or more accoutrements cooperatively interact with the apex of the one or more fitments when substantially aligned.

In illustrative embodiments, the cooperative interaction is selected from snapping, screwing, clamping, adhering, locking, riveting, frictional fitting, bayonetting, or any combination thereof. In illustrative embodiments, the one or more accoutrements are composed of silicone, rubber, vinyl, non-water hardenable urethane, plastic, plastic-based materials, fiberglass, metal, ceramic, polymers, resin, plaster, scented plastic, cellulose, or scented cellulose, or any combination thereof. The one or more accoutrements have a shape selected from polygonal, rectangular, square, circular, curved, concentric, concave, perimetric, diamond, hexagonal, or triangular configurations, or any combination thereof in various embodiments. In some embodiments, the one or more accoutrements are removable or interchangeable, or both. In illustrative embodiments, the one or more accoutrements have a surface, and wherein the surface is decorated, colored, patterned, texturized, designable, colorimetric, modifiable, or designable, or any combination thereof.

In one aspect, the present invention provides methods of adorning an orthopaedic cast that include providing a casting composition entailing at least one layer of reticulated material configured to conform to a body surface that defines a casting region, providing one or more fitments that include a buttress end, an engagement post, and an apex, and perforating the casting composition with the one or more fitments such that the one or more fitments traverse the casting composition while the engagement post remains at least partially embedded, contacting the one or more fitments with one or more accoutrements capable of cooperatively interacting with the one or more fitments, and forming an adorned orthopaedic cast. In various embodiments, the methods further include an engagement post shell to facilitate the perforating step.

In various embodiments, the engagement post shell reversibly envelopes the apex and the engagement post, and where the engagement post shell reversibly contacts an upper surface of the buttress end. In suitable embodiments, the methods further included securing the one or more accoutrements to the apex of the one or more fitments such that the one or more accoutrements and the apex interact and remain connected when the one or more accoutrements are substantially aligned with the apex. In illustrative embodiments, the methods further entail securing the one or more accoutrements to the apex or the engagement post of the one or more fitments such that the one or more accoutrements and the apex or the engagement post reversibly interact when the one or more accoutrements.

In some embodiments, the methods further include excising the apex after the perforating, where the excising exposes an interior hollow region of the engagement post. In various embodiments, the methods further provide for an engagement post shell to facilitate the perforating, where the engagement post shell reversibly envelopes the apex and the engagement post in some embodiments. The engagement post shell reversibly contacts an upper surface of the buttress end in illustrative embodiments as well.

In illustrative embodiments of the present methods, the casting composition is selected from silicone, rubber, vinyl, non-water hardenable urethane, plastic, plastic-based materials, fiberglass, metal, ceramic, one or more polymers, resin, plaster, scented plastic, cellulose, and scented cellulose, or any combination thereof. The body surface is selected from an entire body, lower extremity limbs, upper extremity limbs, one or both shoulders, one or both hips, one or both arms, one or both elbows, one or both hands, one or both wrists, one or both legs, one or both feet, one or both ankles, one or both heels, one or both knees, neck, and head, or any combination thereof.

In illustrative embodiments, the casting region allows for application of the cast to an entire body, lower extremity limbs, upper extremity limbs, one or both shoulders, one or both hips, one or both arms, one or both elbows, one or both hands, one or both wrists, one or both legs, one or both feet, one or both ankles, one or both heels, one or both knees, neck, and head, or any combination thereof. In suitable embodiments, the height of the one or more fitments is from about 0.01 cm to about 10 cm. In various embodiments, the one or more fitments are composed of silicone, rubber, vinyl, non-water hardenable urethane, plastic, plastic-based materials, fiberglass, metal, ceramic, one or more polymers, resin, plaster, scented plastic, cellulose, and scented cellulose, or any combination thereof.

In suitable embodiments, the buttress end, engagement post, and apex of the one or more fitments are integrated to form a single structure. In illustrative embodiments, the buttress end, engagement post, and apex of the one or more fitments are reversibly connected to form a single structure. In some embodiments, the buttress end, engagement post, and apex of the one or more fitments are separate structures capable of reversibly connecting to form a single structure. The one or more fitments perforate the casting composition prior to polymerization of the casting composition in suitable embodiments. The engagement post shell, in certain embodiments, perforates the casting composition prior to polymerization of the casting composition.

In some embodiments, the methods include removing the engagement post shell from the one or more fitments by extracting or unscrewing the engagement post shell from the exterior side of the cast. In illustrative embodiments, the removing allows for the apex of the one or more fitments to remain free of casting composition particulates. In certain embodiments, the one or more fitments are in the stabilized configuration such that the buttress end is about the cast interior, the apex is about the cast exterior, and the engagement post is in an embedded position. In some embodiments, the buttress end comprises an upper surface and a lower surface.

In illustrative embodiments, the upper surface of the buttress end is planar. In various embodiments, the upper surface of the buttress end contacts an interior region of the cast. In some embodiments, the lower surface of the buttress end is convex, while in suitable embodiments, the lower surface of the buttress end possesses one or more adherence nodes, while partially contacting the casting region. When pressure and/or force is applied to the lower surface of the buttress end, in some embodiments, the perforating occurs. In illustrative embodiments, the engagement post is connected to the buttress end and to the apex. In illustrative embodiments, the apex extends beyond the plane of an exterior surface of the cast, while in other embodiments the apex is excised to expose an interior region of the engagement post, which is accordingly flush with an exterior surface of the cast. The interior region of the engagement post is hollow in suitable embodiments.

In certain methodical embodiments, the engagement post has a hollow interior region, where the engagement post is configured to secure the one or more fitments in place. Likewise, in particular embodiments, the one or more accoutrements have an engagement rod that interacts with the hollow interior region of the engagement post. In certain embodiments, the apex has a shape selected from circular, rectangular, coned, pyramidal, grooved, square, polygonal, curved, concentric, concave, perimetric, diamond, hexagonal, or triangular configurations, or any combination thereof.

In some embodiments, the apex is configured to secure the one or more fitments in place. The securing mechanism, in some embodiments, is selected from snapping, screwing, clamping, adhering, locking, riveting, frictional fitting, bayonetting, or any combination thereof. In suitable embodiments, the connecting occurs when the one or more accoutrements cooperatively interact with the apex of the one or more fitments and when the one or more accoutrements are substantially aligned with the apex. In illustrative embodiments, the cooperative interaction is selected from the group consisting of snapping, screwing, clamping, adhering, locking, riveting, frictional fitting, bayonetting, or any combination thereof.

In suitable embodiments, the one or more accoutrements are composed of silicone, rubber, vinyl, non-water hardenable urethane, plastic, plastic-based materials, fiberglass, metal, ceramic, polymers, resin, plaster, scented plastic, cellulose, or scented cellulose, or any combination thereof. In various embodiments, the one or more accoutrements have a shape selected from polygonal, rectangular, square, circular, curved, concentric, concave, perimetric, diamond, hexagonal, or triangular configurations, or any combination thereof. The one or more accoutrements are removable or interchangeable, or both in various embodiments. In illustrative embodiments, the one or more accoutrements have a surface, and wherein the surface is decorated, colored, patterned, texturized, designable, colorimetric, modifiable, or designable, or any combination thereof. And, in some embodiments, the forming is complete when the one or more accoutrements are reversible connected to the apex of the one or more fitments.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE FIGURES

FIG. 1 is a perspective view of an adorned orthopaedic cast of the present invention.

FIG. 2 is a lower-perspective view of a fitment of the present invention.

FIG. 3 is a side-perspective view of a fitment of the present invention.

FIG. 4 is a cross-sectional view of a fitment of the present invention, which includes casting material.

FIG. 5 is a top view of casting materials with an apex of a fitment protruding from the casting material.

FIG. 6 is a top view of casting materials showing a hollow interior region of an engagement post subsequent to apex excision.

FIG. 7 is a bottom view of an accoutrement with engagement rod.

FIG. 8 is a perspective view of an accoutrement engaging an engagement post via an engagement rod.

FIG. 9 is a representative flow chart for methods of the present invention.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

As used herein, unless otherwise stated, the singular forms “a,” “an,” and “the” include plural reference. Thus, for example, a reference to “a cast” or “the cast” includes a plurality of casts.

As used herein, the term “about” will be understood by persons of ordinary skill in the art and will vary to some extent depending upon the context in which it is used. If there are uses of the term which are not clear to persons of ordinary skill in the art, given the context in which it is used, the term “about” in reference to quantitative values will mean up to plus or minus 10% of the enumerated value.

As used herein, the terms “accoutrement”, “accoutrements”, “adornment” or adorned” refer to aesthetic components of the present invention, which functionally interact with a fitment, components thereof, casting components, and the like. For example, an accoutrement is composed of a screw-like engagement rod that cooperatively interacts with a hollow interior region of an engagement post in some embodiments. Likewise, an accoutrement possesses an invagination or cavity region in other embodiments, which securely or reversibly interacts, connects, snaps, screws, and the like, onto an apex protruding from a surface region of a cast in some embodiments.

As used herein, the terms “apex” or “apical region” as referred to in the context of one or more fitments of the present invention, impart a region at the pinnacle or top of a fitment, which, at least initially, projects beyond an upper surface of an orthopaedic cast, e.g., in embodiments where the apex is not excised (or prior to the excision or removal of the apex). In illustrative embodiments, the apex cooperatively interacts with a cavity region or invagination of an accoutrement. The apex, however, is excised in certain embodiments to provide access to a hollow interior region of an engagement post, i.e., when, for example, an accoutrement possesses an engagement rod. The apex may be an anti-retropulsion apex insofar as it is configured to protrude or project beyond a cast region surface without being pushed back, e.g., by an accoutrement, pushing back and/or retracting into the casting materials. Non-limiting embodiments of an anti-retropulsion apex is shown in the figures provided herein.

As used herein, the terms “body surface” or “casting region,” refer to a part or structure of the body, having a certain function or functions, which can provide an area for cast immobilization, and includes, but is not limited to, an entire body, lower extremity limbs, upper extremity limbs, one or both shoulders, one or both hips, one or both arms, one or both elbows, one or both hands, one or both wrists, one or both legs, one or both feet, one or both ankles, one or both heels, one or both knees, neck, and head, or any combination thereof. In some embodiments of the present invention, the “casting region” allows for application of the cast to an entire body, lower extremity limbs, upper extremity limbs, one or both shoulders, one or both hips, one or both arms, one or both elbows, one or both hands, one or both wrists, one or both legs, one or both feet, one or both ankles, one or both heels, one or both knees, neck, and head, or any combination thereof.

As used herein, the term “buttress end” refers to a fitment region or separate component of a fitment that is distal to the apex of the fitment. The buttress end possesses an upper surface and a lower surface, which are respectively planar and convex in certain embodiments. In some embodiments, the buttress end is a solid constituent of a fitment, while in other embodiments the buttress end possesses an open (hollow) region in fluid communication with a hollow interior region of an engagement post. The buttress end is proximal to an engagement post of a fitment, for example, which can be reversibly attached thereto and/or may consist of a singular structure.

As used herein, the terms “cast”, “casting”, “casting materials”, “orthopaedic cast”, and/or “orthopedic cast” used in the context of preparation, production, and/or methods of making an immobilization substrate suitable for immobilizing a particular region or regions of an individual's anatomy, refer to any surface or structure created that is capable of immobilizing a body surface or region and any ancillary regions coterminous therewith. Such casts have various contemplated surfaces, and/or are composed of materials, which include, but are not limited to, silicone, rubber, vinyl, non-water hardenable urethane, plastic, plastic-based materials, fiberglass, metal, ceramic, polymers, resin, plaster, scented plastic, cellulose or scented cellulose, stocking materials, padding, stockinettes, biocompatible polymers, biodegradable polymers, copolymers, and the like.

As used herein, the terms “cooperatively interact” or “cooperatively interacting” or “cooperative interaction” refer to the association of two or more adjoining components, where each component functions to facilitate the association. For example, a fitted accoutrement cooperatively interacts with a component that the accoutrement was fabricated to fit.

As used herein, the terms “disengage”, “reversibly disengage”, “reversibly disengaged”, and “disengaged configuration”, all refer to the act or state of no longer being securely associated or connected. For example, two components are disengaged with each other they are not in contact with each other. However, such components can be in contact while concomitantly occupying a disengaged state. In this circumstance, the components would not be securely engaged by such means as, for example, a locking mechanism. If such components are “reversibly disengaged” then the components are capable of engaging at a different time.

As used herein, the terms “engage”, “reversible engage”, “reversibly engaged”, and “engaged configuration” all refer to the act or state of being associated or connected in a secure manner for the purpose of joining two or more components for a period of time. For example, two components are engaged with each other when they are in contact and securely connected or associated for a period of time. To be in the engaged state, the components are in contact while concomitantly occupying an engaged state, such as, for example, a locked state. If such components are “reversibly engaged” then the components can be engaged and disengaged with respect to the features enabling such association and disassociation, respectively.

As used herein, the term “engagement post” refers to a solid, hollow or partially hollow tube or longitudinal member configured as, e.g., poles, members, tubes, plugs, screws, caps, rivets, covers, keys, wrenches, gripping members, and the like, or any combination thereof. A hollow interior region of an engagement post, in some embodiments, is capable of cooperatively interacting with an engagement rod of an accoutrement in some embodiments. The engagement post also includes an engagement post shell in some embodiments.

As used herein, the term “engagement post shell” refers to, e.g., caps, covers, seals, and the like, or any combination thereof, such that the engagement post shell substantially covers the region or area intended to be covered, e.g., an apex, engagement post and/or a region thereof or the entire fitment. A variety of shell shapes and sizes are provided herein, and include, but are not limited to, for example, shapes such as, e.g., polygonal, rectangular, square, circular, curved, concentric, concave, perimetric, diamond, hexagonal, or triangular configurations, or any combination thereof.

As used herein, the term “fitment” refers to a structure composed of, but not limited to, a buttress end, an engagement post, and an apex, where each of the components are conjoined to form a singular structure in suitable embodiments. In some embodiments, the fitment is composed of a buttress end, an engagement post, and an apex that reversible interact, connect, engage, lock, secure, rivet and/or disconnect, disengage, unlock, and/or remove from, for example, each other and/or an adornment of the present invention. Such interaction typically occurs through the engagement post and/or the apex in illustrative embodiments. A fitment of the present invention, moreover, includes an engagement post shell in various embodiments.

Apical fitment regions, i.e., an apex, provided herein are configured to accesses a port, portal, region, or complementary fitting, provided in an accoutrement in some embodiments. In accord, the apex is positioned within an accoutrement, when cooperatively interacting, in a manner selected from angled, straight, slanted, tapered, curved, diagonal, random, polygonal, rectangular, square, circular, curved, concentric, concave, perimetric, diamond, hexagonal, or triangular configurations, or any combination thereof. Likewise, for example, the apex is capable of reversibly interacting, engaging, connecting, locking, and/or securing to an accoutrement invagination and/or cavity in suitable embodiments.

Moreover, the engagement posts of the present invention may be configured as, e.g., solid tubular structures, hollow or partially hollow tubular structures, solid poles, members, plugs, screws, caps, rivets, covers, keys, wrenches, gripping members, and the like, or any combination thereof. Likewise an accoutrement possesses an engagement rod in some embodiments, which is capable of cooperatively interacting with a hollow interior region of an engagement post in some embodiments. The engagement post also includes an engagement post shell in some embodiments, as further defined herein. Buttress end components of the one or more fitments described herein are noted above.

As used herein, the term “reticulated material” refers to compositions or composition matrices composed of network constituents forming one or more layers or matrix configurations. For example, reticulated material include, but are not limited to, fiberglass, silicone, one or more polymers, plastic, resin, plaster, stocking material, and padding, and combinations thereof. Examples of casting polymers are generally known in the art.

As used herein, the terms “substantial” or “substantially” within the context of a “substantially enveloped” surface or region or a “substantially aligned” configuration, refer to, e.g., total or complete envelopment or alignment, and the like, but also includes lesser than complete or total envelopment or alignment, and the like, insofar as the intended purpose for performing the act can be carried out to the same extent as if the, e.g., envelopment or alignment, were total or complete.

General Overview

Medical intervention is required when damage to the bone occurs, such as, e.g., fracturing, separating, and breaking. In this regard, the use of orthopaedic casts are ubiquitous with respect to limb immobilization associated with such bone fractures and breaks, while also providing medical practitioners with a means for treating, e.g., musculoskeletal conditions and osteodegeneration. In contrast to flexible splints, which are non-circumferential immobilizers that accommodate swelling and inflammation, orthopaedic casts are ridged circumferential applications capable of immobilizing any region of the body. Such casts, however, can be associated with complications stemming from the lack of flexibility and are therefore reserved for complex and/or definitive bone fracture management. Along the same lines, excessive immobilization can lead to chronic pain, joint stiffness, muscle atrophy, or more severe complications, e.g., complex regional pain syndrome. Patients that are placed in a cast accordingly require regular monitoring to ensure compliance and proper recovery. See, e.g., Boyd et al., “Splints and Casts: Indications and Methods.”Am. Fam. Physician. 80(5):491-499 (2009).

Compliance, however, can be improved by aesthetically enhancing the appearance of an orthopaedic cast, alone or in combination with other considerations, e.g., proper comfort and patient education. The present invention accordingly concerns novel orthopaedic casts, orthopaedic cast components, and systems and methods for making such casts and components to ensure proper bone healing via non-disruptive cast applications, which comport with a patient's aesthetic preferences. As such, the present invention relates to, inter alia, adorned casts including, but not limited to, adorned orthopaedic casts, fitments and/or fitment components, and associated functionalities. Taken together, the foregoing constituents, and the invention as provided herein, in toto, allow, facilitate, enhance, and/or improve an individual's experience associated with cast immobilization. Such aspects of the present invention consequently provide for increased compliance, comfort and aesthetic enhancement while wearing a cast.

### Orthopaedic Casts And Components Introduction

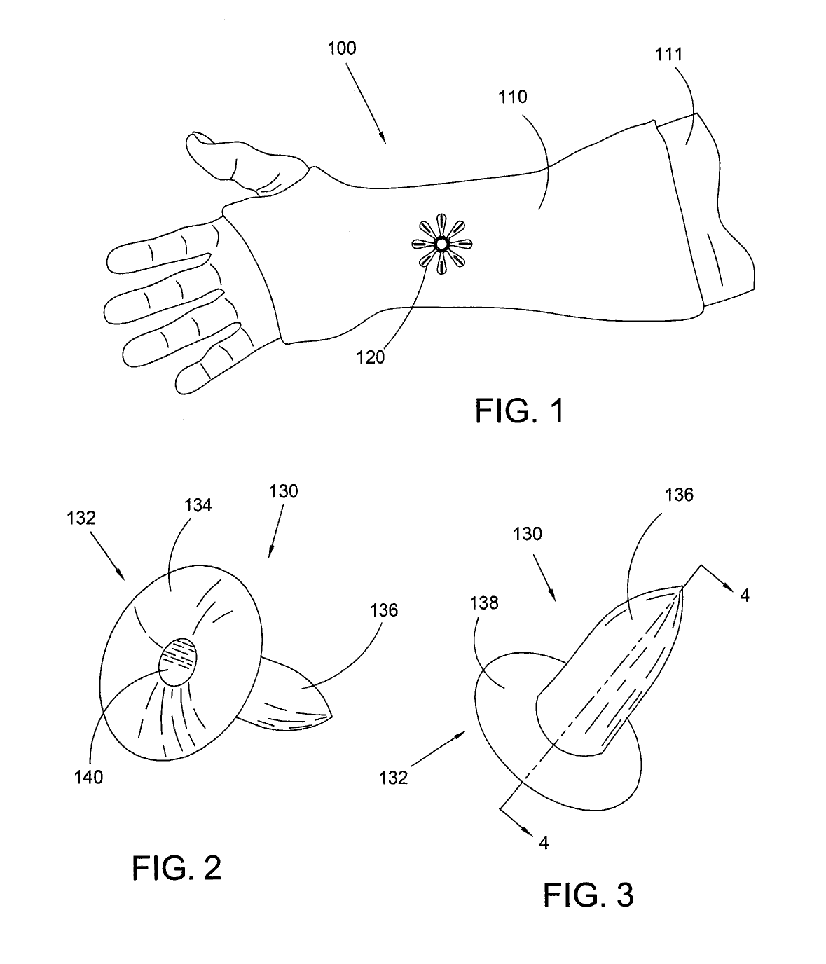

The one or more fitments of the present invention are configured in suitable embodiments as small diameter cast accessories that are inserted into, and at least partially perforate through, casting materials. Furthermore, the fitments reversibly engage a separate-decorative-accoutrement, which is interchangeable with various other accoutrement varieties in illustrative embodiments. These decorative features entail a variety of colors, patterns and designs, and are composed of, for example, an assortment of materials, e.g., plastic, rubber, resins, etc., as further detailed herein. Reverting to the fitments, these features of the present invention include, without limitation, (i) a buttress end, i.e., the region to which force is applied when penetrating a fitment through casting materials, (ii) a tubular engagement-post, which possesses (iii) an anti-retropulsion apex for securement to the cast exterior in illustrative embodiments. The apex, however, is subsequently excised in certain embodiments as further detailed below. And (iv) an engagement-post shell that facilitates engagement-post penetration through the casting material. See FIGS. 1-8.

Briefly, methods and system components of the present invention involve one or more fitments that are provided to the interior surface of casting materials, i.e., that are positioned about a casting region. By applying force to the buttress end, a fitment is impelled through the casting material, e.g., to which a guide hole was previously formed in some embodiments. In other embodiments, no guide hole is necessary. The engagement post traverses the casting material, in various embodiments, such that the apex breaches the exterior layer or surface of the casting material. As noted, the apex possesses anti-retropulsion physiognomies, i.e., which prevent the fitment from retracting once the apical region has completely breached the casting material exterior, in various embodiments. In accord, the accoutrements possess an invagination or cavity that facilitate cooperative interaction with the apex in illustrative embodiments.

In other embodiments, an apex that has breached the casting material surface is removed by excision. Here, the engagement post is typically hollow, e.g., possessing a central longitudinal cavity for receiving, e.g., an engagement or securement rod, which is attached to, or part of, an accoutrement in certain embodiments. Excision of the apex exposes a hollow interior region of an engagement post, which, in some embodiments, possesses a superior face that is flush with the surface of the casting materials. The accoutrements to this end possess a securement or engagement rod capable of cooperatively interacting with the exposed portion, including the superior face, of the engagement post. Such interaction may occur via plugging, screwing, capping, covering, sealing, corking, locking, riveting, and/or stopping, or any combination thereof. In illustrative embodiments, the one or more accoutrements are removable or interchangeable, or both. Suitable embodiments, moreover, provide for an engagement post shell that is removed from the fitment via the exterior surface of the casting materials, i.e., subsequent to facilitating engagement post perforation.

Furthermore, the adorned casts of the present invention include a casting composition with at least one layer of reticulated material configured to conform to a body surface that defines a casting region, where the casting region is substantially enveloped by the casting composition to form an orthopaedic cast of the present invention. Such reticulated materials include, but are not limited to, for example, silicone, rubber, vinyl, non-water hardenable urethane, plastic, plastic-based materials, fiberglass, metal, ceramic, polymers, resin, plaster, scented plastic, cellulose or scented cellulose, or any combination thereof.

In some embodiments, the body surface is an entire body, lower extremity limbs, upper extremity limbs, one or both shoulders, one or both hips, one or both arms, one or both elbows, one or both hands, one or both wrists, one or both legs, one or both feet, one or both ankles, one or both heels, one or both knees, neck, and head, or any combination thereof. In illustrative embodiments, the casting region allows for application of the cast to an entire body, lower extremity limbs, upper extremity limbs, one or both shoulders, one or both hips, one or both arms, one or both elbows, one or both hands, one or both wrists, one or both legs, one or both feet, one or both ankles, one or both heels, one or both knees, neck, and head, or any combination thereof.

Selection of a specific cast type nevertheless varies in accord with the body region being treated, and on the acuity and stability of the injury. Indications and accurate application techniques also vary and are commonly encountered in a primary care setting. Typically, casting begins by placing the injured extremity in its functional position. Subsequently, in accord with embodiments of the present invention, padding and/or stockinette application occurs followed by circumferential application of from about 1, 2, 3, 4, 5, 6, 7, 8, 9, 10, to about from 2, 3, 4, 5, 6, 7, 8, 9, 10, 11, 12, 13, 14 or 15 layers of cotton padding or stockinette material. Circumferential application of casting materials or composition, e.g., plaster or fiber-glass, and the like, are then added. Typically, about 2-inch padding is used for hand applications and from about 2- to 6-inch padding for upper extremities, where about 3-inch padding and from about 4- to 6-inch padding are employed for foot and lower extremities applications, respectively.

Examples of upper extremity casts, lower extremity casts, spica thumb casts, short arm casts, and the like, are described in U.S. Provisional Patent Application No. 61/756,076, which is herein incorporated by reference in its entirety. See also, e.g., Eiff et al., eds, Fracture Management for Primary Care. 2nd ed. Philadelphia, Pa.: Saunders; 1-70 (2003).

### Adorned Casts, Systems And Methods

In one aspect, the present invention provides for an adorned cast entailing a casting composition as described above, e.g., possessing at least one layer of reticulated material configured to conform to a body surface that defines a casting region, where the casting region is substantially enveloped by the casting composition to form an orthopaedic cast; one or more fitments at least partially embedded in the orthopaedic cast, where the one or more fitments are composed of the components noted herein, e.g., a buttress end, an engagement post, and an apex; and one or more accoutrements capable of cooperatively interacting with the one or more fitments.

An engagement post shell is further provided in various embodiments, which reversibly envelopes the apex and the engagement post, and, in illustrative embodiments, reversibly contacts an upper surface of the buttress end. See FIGS. 2-8. The fitments and components thereof may be composed of silicone, fiberglass, metal, ceramic, one or more polymers, rubber, resin, plaster, plastic, scented plastic, cellulose, or scented cellulose, scented polymers, or any combination thereof, in some embodiments. Scented materials, such as, e.g., scented plastic, scented polymers and cellulose, are materials that are currently available for use in casting applications in suitable embodiments.

The engagement post connects a buttress end and an apex of the one or more fitments of the present invention, all of which are integral components forming a single configuration or structure in certain embodiments. In other embodiments, each fitment component is a separate member capable of interacting, securely or reversibly, to form a multiplex component structure. See FIGS. 2-3. As shown in FIG. 2, the buttress end, as contacting or connected to an engagement post, may possess an opening in fluid communication with a hollow interior region of an engagement post in certain embodiments. The engagement post is nevertheless a solid member in other embodiments. Regardless of the structural configurations concerning the one or more fitments, a central objective of the fitment and/or fitment components relates to receiving and/or engaging one or more accoutrements. This cooperative interaction occurs when, for example, one or more accoutrements are substantially aligned with the fitment, e.g., the apex or the hollow interior region of the engagement post.

Engagement posts of the present invention constitute tubular, semi-tubular, elongated or compressed conduits, members, regions, channel passages, tubes, and the like, in suitable embodiments. In this regard, the engagement posts are capable of interacting, connecting, engaging, locking, securing, riveting and/or disconnecting, disengaging, unlocking, and/or removing from a buttress end, apex, engagement rod and/or other components or materials of the present invention. In some embodiments, the engagement post is a section of a single integrated article, where the article is a fitment in suitable embodiments.

In illustrative embodiments, the engagement posts are configured to access or function as a port, portal, region, or complementary fitting for cooperatively interacting with an engagement rod, an accoutrement, an accoutrement having an engagement rod, and the like, in some embodiments. In accord, the engagement posts are positioned within the casting materials and/or with respect to any of the fitment components in a manner selected from angled, straight, slanted, tapered, curved, diagonal, random, polygonal, rectangular, square, circular, curved, concentric, concave, perimetric, diamond, hexagonal, or triangular configurations, or any combination thereof, such that they are capable of reversibly interacting, engaging, connecting, locking, and/or securing to the fitment components or to accoutrements as provided herein.

Such positioning reversibly secures the engagement post to the apex, buttress end and/or the one or more accoutrements via a plugging, screwing, capping, covering, sealing, corking, locking, riveting, and/or stopping mechanism, or any combination thereof in some embodiments. In suitable embodiments, an engagement post is removable and/or interchangeable with other engagement posts and/or other components of the present invention, e.g., an engagement rod. As such, in some embodiments, an engagement post is interchangeable with, or identical to, an engagement rod. Moreover, the engagement posts may be configured as, e.g., hollow, partially hollow, or solid structures, such as, but not limited to rods, poles, members, tubes, plugs, screws, caps, rivets, covers, keys, wrenches, gripping members, and the like, or any combination thereof.

In addition to the various shapes and sizes of the engagement posts, a variety of configurations are provided for the one or more buttress ends, apical regions, engagement post shells, one or more fitments, accoutrements, and/or any other component of the present invention, which include, but are not limited to, for example, shapes such as, e.g., polygonal, rectangular, square, circular, curved, concentric, concave, perimetric, diamond, hexagonal, or triangular configurations, or any combination thereof.

In illustrative embodiments, the height and/or width of the one or more fitments (as a whole or for each constituent component thereof, individually) is from about 0.001, 0.01, 0.1, 0.5, 0.75, 1, 2, 3, 4, or 5 cm to about from 0.01, 0.1, 0.5, 0.75, 1, 2, 3, 4, 5, 6, 7, 8, 9, 10, 15, 20, 25 or 30 cm. More specifically, some embodiments of the present invention provide for fitments and/or components thereof having a height and/or width from about 0.1, 0.5, 0.75, or 1 cm to about from 0.5, 0.75, 1, 2, or 3 cm. In suitable embodiments, the height and/or width is from about 0.01 cm to about 10 cm, while in illustrative embodiments, the height and/or width is from about 2 cm to about 4 cm.

The full diameter of the one or more fitments is measured vis-à-vis the buttress end, in some embodiments, and is from about 0.001, 0.01, 0.1, 0.5, 0.75, 1, 2, 3, 4, or 5 cm to about from 0.01, 0.1, 0.5, 0.75, 1, 2, 3, 4, 5, 6, 7, 8, 9, 10, 15, 20, 25 or 30 cm. Other embodiments include a diameter from about 0.1, 0.5, 0.75, or 1 cm to about from 0.5, 0.75, 1, 5, or 10 cm. In suitable embodiments, the diameter is from about 1 cm to about 10 cm, while in illustrative embodiments, the diameter is from about 4 cm to about 6 cm. Buttress ends of the present invention further include adherence nodes that directly contact a body surface or stockinette material while at least functioning to secure, anchor, position and/or adhere the fitment to the surface being treated. In some embodiments, the nodes have shapes selected from conical, frusto-conical, rounded, flat, pointed, burred, bubble-shaped, dome-shaped, circular, rectangular, square, polygonal, curved, concentric, concave, perimetric, diamond, hexagonal, or triangular configurations, or any combination thereof. The adherence nodes can be of any shape or size to the extent that such shapes and sizes provide for the intended purpose, as described herein.

Subsequent to perforating, penetrating, and/or impelling through a casting material surface, as more fully detailed below, the apex extends beyond the plane of an exterior surface of the casting materials in certain embodiments. In this configuration, the apex functions as a protuberance for connection or interaction with an accoutrement of the present invention, which accordingly possesses a complementary region, e.g., cavity or invagination, capable of securely or reversibly interacting with the protuberance. Such interactions may occur via, but are not limited to, plugging, screwing, capping, covering, sealing, corking, locking, riveting, and/or stopping, or any combination thereof. In some embodiments, the one or more accoutrements are removable or interchangeable, or both.

Concerning fitment or apex interaction with an accoutrement, certain embodiments impart an engaged configuration when the component, e.g., apex, is substantially parallel to an axis of the one or more accoutrements. In contrast, the apex (or other fitment components) is in a substantially orthogonal position to an axis of the one or more accoutrements when in the disengaged configuration. Other embodiments of the present invention provide for engagement, reversibly or permanently, via cooperative interaction, such as, for example, screwing, clamping, adhering, locking, riveting, friction fitting, or any combination thereof. In illustrative embodiments, the cooperative interaction, reversible engagement, and/or securing is selected from snapping components, screws, clamps, rivets, adhesives, locks, friction fitting, plugs, caps, covers, seals, rivets, corks, and stoppers or any combination thereof.

After the apical protuberance has formed with respect to the casting material surface, however, certain embodiments entail excision or removal of the apex, i.e., the apical region forming the protuberance, via methods known to the skilled artisan. Under these circumstances, for example, the excision exposes an interior hollow region of an engagement post in some embodiments, where the upper portion, i.e., the superior face, is flush with the exterior surface of the casting materials. In illustrative embodiments, the engagement post accordingly possesses a hollow interior region capable of receiving an engagement rod, for example, which, is capable of cooperative interaction with an accoutrement in some embodiments. Simply put, the engagement rod cooperatively interacts with the engagement post, via the hollow interior, of the one or more fitments when the apex has been excised pursuant to suitable embodiments of the present invention.

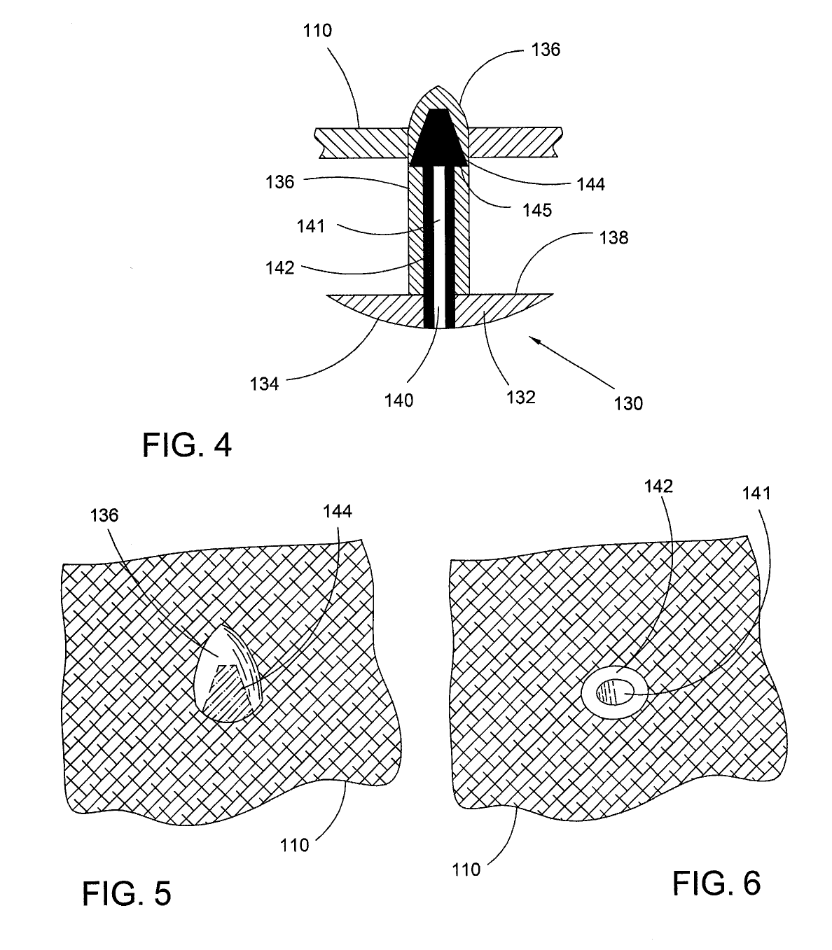

Certain embodiments nevertheless provide for an apex—accoutrement interaction, i.e., where the apex is not excised, while further entailing mechanisms for preventing retraction, e.g., of the apex, back into the casting materials after the initial perforation has formed. In this respect, certain embodiments of the present invention provide for an anti-retropulsion apex, oriented to prevent, or at least minimize the potential for, retraction or ejection of the one or more fitments or fitment components, e.g., the apex, from the casting materials. The anti-retropulsion apex is positioned at the apical region of the fitment in illustrative embodiments, thereby preventing retropulsion of the fitment in any direction. See FIGS. 4-5.

In concert with the adorned casts, fitments and components thereof, as provided herein, systems and methods for adorning such casts are similarly within the scope of the present invention. In one aspect, such systems include a casting composition to form an orthopaedic cast; one or more fitments, e.g., including a buttress end, engagement post, and an apex, where the engagement post connects the buttress end to the apex such that when force is applied to the buttress end the apex is impelled through the casting material; and one or more accoutrements that cooperatively interact with the one or more fitments to form an adorned orthopaedic cast.

The present invention similarly provides methods of adorning orthopaedic casts by providing a casting composition including at least one layer of reticulated material configured to conform to a body surface that defines a casting region, while also providing one or more fitments as detailed above, perforating the casting composition with the one or more fitments such that the one or more fitments traverse the casting composition while the engagement post remains at least partially embedded therein, contacting the one or more fitments with one or more accoutrements capable of cooperatively interacting with the one or more fitments, and thereby forming an adorned orthopaedic cast. An engagement post shell is also provided in various systematic and/or methodical embodiments to facilitate application of the one or more fitments, e.g., through the casting materials.

Prior to employing the fitments and fitment components of the present invention, however, it is important to regard threshold considerations associated with patient cast application, i.e., with respect to the methods and systems of the present invention. In this regard, orthopaedic immobilization assemblies, such as casts, splints, and/or supports, typically employ materials that form, manifest, strengthen, solidify, harden, shape, congeal, polymerize, and the like, via use of water (or other catalyst) catalyzed processes, e.g., adding water and/or allowing for such materials to form for a certain period of time. In practice, casting materials may be impregnated with a water hardenable urethane, and, at the time the cast or support is to be applied to the patient, it is dipped into water, applied to the injured part of the anatomy, and, within minutes to hours, solidifies. See, e.g., EP 1372549, which is incorporated herein by reference in its entirety. In some embodiments of the present invention, the foregoing process occurs in the presence of one or more fitments and/or components thereof.

The reticulated cast material and/or casting composition to be applied to a patient is selected from silicone, rubber, vinyl, non-water hardenable urethane, plastic, plastic-based materials, fiberglass, metal, ceramic, polymers, resin, plaster, scented plastic, cellulose or scented cellulose or any combination thereof in some embodiments. Such compositions allow for methodical wrapping, placing, making, and/or circumferentially applying the casting material to a body surface.

Orthopaedic casts of the present invention typically form, after application to a patient, in minutes, hours, days, or weeks. In some embodiments, the cast forms in about from 0.1-100, 0.5-90, 1-80, 1.5-70, 2-60, 2.5-55, 3-50, 3.5-40, 4-30, 4.5-20, 5-15, or 6-7 seconds minutes, hours, or days. In illustrative embodiments, the cast forms in about from 0.1-2 hours. It will be readily apparent to the skilled artisan that numerous additional variables can affect cast formation in the presence or absence of one or more fitments or fitment components. Typical materials which may be used are silicone, rubber, adhesive, foam, TPE, vinyl, non-water hardenable urethane, and other plastic materials, such as, for example, fiberglass, Kevlar®, or aramids, for examples, and to materials having comparable high strength, as discussed in U.S. Pat. Nos. 6,139,513, and 6,186,966, which are incorporated herein by reference in their entirety. See, e.g., EP 1372549 and EP 0755664, which are incorporated herein by reference in their entirety.

Bonding material is also provided in some embodiments in the presence or absence of comfort foaming material or other applications as described above. The bonding material may be applied in a variety of ways, such as, for example, by spraying, brushing, pressing, dipping or the like, before or after application of the casting material to the casting region. See, e.g., EP 1372549, which is incorporated herein by reference in its entirety. Depending on the bonding material chosen and the method of application, it is possible that adjustments, manipulations, reconfigurations, and the like, can be performed. Forming the guide holes and/or placing the one or more fitments or fitments components in the cast before, during, or after cast application is also an aspect of the present methods, whereas, in suitable embodiments, casting material is applied after the one or more fitments or fitments components have been positioned on the patient. Accordingly, such components will be readily incorporated into the cast as it forms, hardens, and/or solidifies, as further described below.

Prior to, or simultaneous with, casting material solidification, drying, polymerization, and/or hardening, however, illustrative embodiments of the present invention provide for the application of mechanical or manual force to the lower surface of a buttress end of the one or more fitments of the present invention. Consequently, the fitment is impelled through the casting materials such that the fitment and/or fitment components are partially embedded in the casting materials. The fitments are then contacted with one or more accoutrements capable of cooperatively interacting with the fitments, e.g., via an apex—accoutrement or an engagement post—accoutrement interaction, which thereby forms an adorned orthopaedic cast. While the foregoing steps provide for, in part, the production of an adorned cast, other embodiments serve to facilitate such methods and systems by achieving a variety of adorned cast products, applications and configurations, for example.

Along the same lines, some embodiments of the present methods and systems further include an engagement post shell to facilitate perforation of the casting materials by the one or more fitments or components thereof. In suitable embodiments, the engagement post shell reversibly envelopes the engagement post, an upper surface of the buttress end, and the apex, while also reversibly contacting an upper surface of the buttress end in some embodiments. Likewise, the fitment apex is covered by the engagement post shell in suitable embodiments. As such, when the shell-covered apex breaches the casting material surface, the superior region of the engagement post shell is presented, which can be readily gripped via digital manipulation or by using tools, medical or conventional, well known in the art, e.g., pliers, prongs, forceps, and the like. The engagement post shell is subsequently pulled through the casting materials via the previously formed perforation, which concomitantly removes it from the fitment in some embodiments. Accordingly, the remaining fitment components are free of any particulate material.

In turn, illustrative embodiments of the present invention further provide for reversibly securing the one or more accoutrements to the apex, as described above, or to the engagement post of the one or more fitments. In this respect, the one or more accoutrements and the engagement post reversibly interact thereby allowing the patient to change accoutrements as desired. The methods further entail excising the apex after the perforating step, where the excising exposes an interior hollow region of the engagement post. Accordingly, an accoutrement possessing an engagement rod is positioned to cooperatively interact with the exposed region, i.e., via substantial alignment as further described herein. In suitable embodiments, excision of the apex imparts an exposed region—the superior face—of the engagement post that is flush with an exterior surface of the cast. This exposed region, in illustrative embodiments, reveals an interior hollow region of an engagement post that is configured to securely or reversibly receive one or more accoutrements having an engagement rod.

Likewise, the present methods involve attaching, contacting, and/or securing the one or more accoutrements to the one or more fitments or fitment components by cooperatively interacting, e.g., combining, complementary components, such as, for example, an accoutrement and an apex. Cooperative interactions occurs in embodiments where the accoutrements are substantially aligned with the one or more fitments or fitment components. Methods of the present invention further include securing the fitments or fitment components to the accoutrements such that the fitments or fitment components and the accoutrements interact and remain connected when substantially aligned. The skilled artisan will understand that the foregoing features may be interchanged with similar objects known in the art. Additionally, the present invention contemplates the interchangeability of individual features of the present invention with respect to the same features, albeit in modified embodiments.

### Accoutrements

Aesthetic considerations, such as, for example, appearance, texture, entertainment and the like, which concern an individual when cast immobilization is required for extended (or short) periods of time, are provided in illustrative embodiments of the present invention as described above and further detailed as follows. Indeed, the present invention provides a plethora of features for enhancing and improving the cast wearing experience. As such, illustrative embodiments of the present invention impart one or more accoutrements as decorative ornamental objects having a surface and/or design that are decorated, colored, patterned, texturized, designable, colorimetric, modifiable, or designable, or any combination thereof. Examples of surface or design configurations and/or ornamental objects that may be attached include, but are not limited to, charms, accessories, entertainment applications, aesthetic components, jewelry, stickers, adhesives, decals, ornaments, styling elements, watch features, watches, calculators, mobile phone accessories, computer and electronic application accessories, holders, make-up and beauty applications, learning and/or educational applications, sports and trading card applications, and the like, all of which are interchangeable and accessible to an individual wearing the orthopaedic cast of the present invention. Such surface configurations and/or objects may also embody, but are not limited to, animals, cars, personalities, structures, words, items, apparel, jewelry, and the like.

Referring now to the illustrations, FIG. 1 is a perspective view of an adorned orthopaedic cast embodiment 100. The cast 110 is circumferentially positioned around the hand and arm 111, while accoutrement 120 (flower shape) is shown. FIG. 2 depicts a lower-perspective view of an embodied fitment 130 of the present invention. Fitment 130 includes buttress end 132 with lower surface 134 which defines a contact region (for applying force), where, in suitable embodiments, buttress end 132 has an opening 140 in fluid communication with a hollow interior region of an engagement post (not shown). Adherence nodes (not shown) are oriented on lower surface 134 such that they prevent, or at least minimize the potential for, shifting of fitment 130. Engagement post shell 136, covering the engagement post and apex (not shown) is also provided. FIG. 3 is a side-perspective view of an embodied fitment 130 of the present invention. Buttress end 132 upper surface 138 is juxtaposed to engagement post shell 136.

FIG. 4 shows a cross-sectional view of an embodied fitment 130 traversing cast 110. Buttress end 132, lower surface 134, and upper surface 138 are shown with engagement post shell 136 provided, which envelopes engagement post 142 (solid region) and solid apex 144. Buttress end opening 140 continues into hollow interior region 141 of engagement post 142. Solid apex 144 with anti-retropulsion head 145, are detailed as shown. FIG. 5 is a top view of fitment 130 with engagement post shell 136 covering apex 144 (hidden lines) projecting from an exterior surface of cast 110. FIG. 6 depicts the solid (142) and hollow (141) regions of an engagement post following removal of engagement post shell 136 and apex 144 excision. Hollow interior region 141 is configured to receive an engagement rod of an accoutrement (not shown).

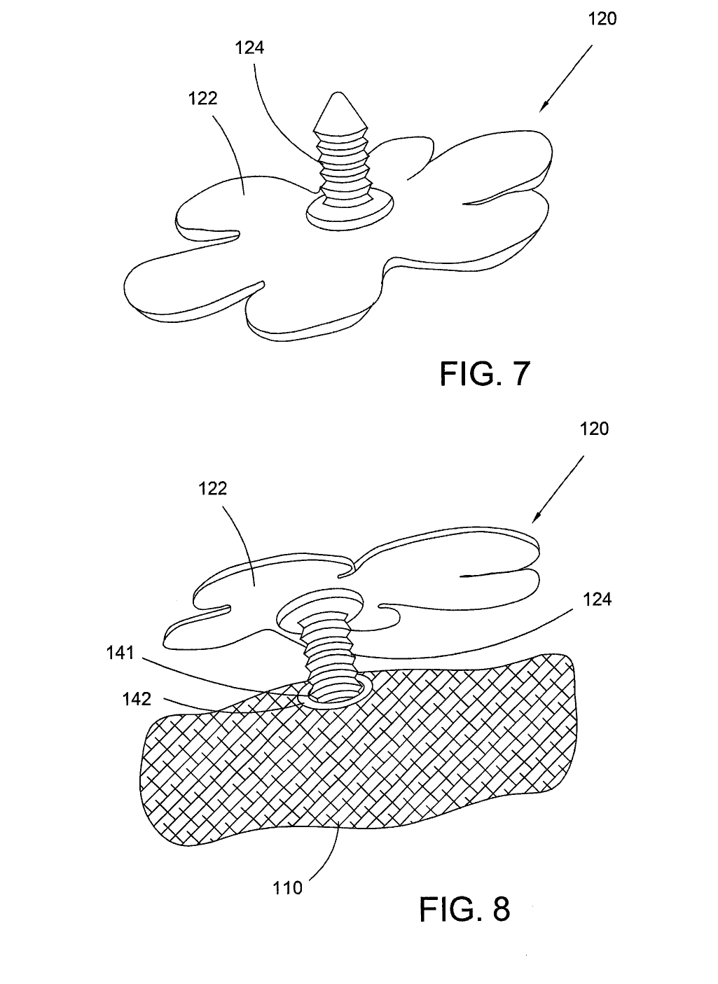

As shown in FIG. 7, a bottom perspective view of accoutrement 120 with inferior surface 122, and engagement rod 124 which protrudes from, and is connected to, accoutrement 120. Engagement rod 124 cooperatively interacts with hollow interior region 141 of engagement post 142 via a screwing mechanism (see FIG. 4), which imparts a mechanism for engagement and/or securement as shown in the perspective view of FIG. 8. The skilled artisan readily understands that the foregoing features may be interchanged with similar objects known in the art. Likewise, the present invention contemplates the interchangeability of individual features of the present invention with respect to the same features, albeit in modified embodiments, e.g., various accoutrements may be interchanged in accord with various complementary fitments components.

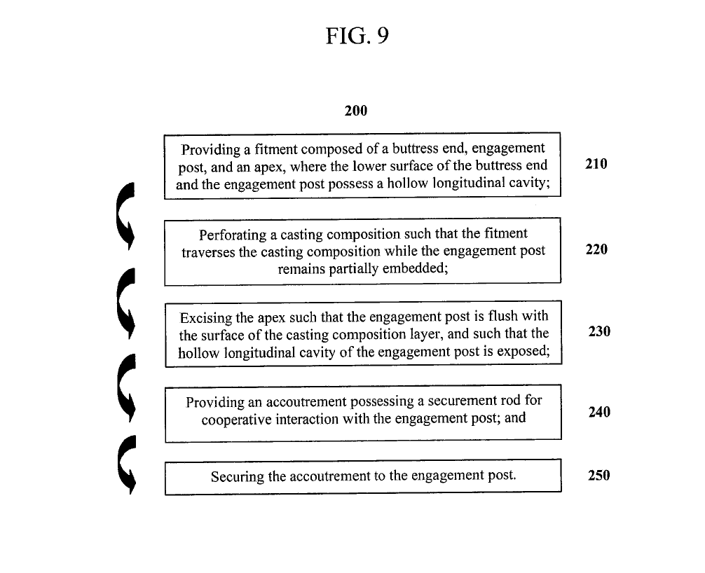

FIG. 9 shows diagrammatic flow-chart 200 representing one process for making an adorned orthopaedic cast of the present invention. Reticulated material is provided as the casting composition, which is configured to conform to a body surface defining a casting region. In operation 210, a fitment composed of a buttress end, engagement post, and an apex is provided, where the lower surface of the buttress end has an opening in fluid communication with a hollow longitudinal cavity of an engagement post. Operation 220 entails perforating a casting composition with a fitment, such that the fitment traverses the casting composition while the engagement post remains partially embedded. The apex is excised in operation 230 and therefore exposes the superior region of a hollow engagement post, which is consequently flush with the surface of the casting composition layer. An accoutrement possessing an engagement rod is provided in operation 240, which functions to cooperatively interact with the engagement post. Operation 250 denotes completion of one methodical embodiment after the accoutrement is reversibly secured to the engagement post. See FIG. 9.

What is claimed is:

1. An adorned cast, comprising:
(a) a casting composition comprising at least one layer of reticulated material configured to conform to a body surface that defines a casting region, wherein the casting region is substantially enveloped by the casting composition to form an orthopaedic cast;
(b) one or more fitments at least partially embedded in the orthopaedic cast, wherein the one or more fitments comprise a buttress end, engagement post, and an apex; and
(c) one or more accoutrements capable of cooperatively interacting with the one or more fitments.

2. The adorned cast of claim 1, further comprising an engagement post shell.

3. The adorned cast of claim 2, wherein the engagement post shell reversibly envelopes the apex and the engagement post, and wherein the engagement post shell reversibly contacts an upper surface of the buttress end.

4. The adorned cast of claim 1, wherein the engagement post connects the buttress end and the apex, and wherein the engagement post is solid or comprises a hollow interior region.

5. The adorned cast of claim 1, wherein the apex extends beyond the plane of an exterior surface of the orthopaedic cast, or wherein the apex is excised leaving the fitment flush with the exterior surface of the orthopaedic cast.

6. The adorned cast of claim 1, wherein the one or more accoutrements cooperatively interact with the apex of the one or more fitments when the one or more accoutrements are substantially aligned with the apex.

7. The adorned cast of claim 1, wherein the one or more accoutrements further comprise an engagement rod, wherein the engagement rod cooperatively interacts with the one or more fitments when the apex has been excised.

8. A system for adorning an orthopaedic cast, comprising:
(a) a casting composition to form the orthopaedic cast;
(b) one or more fitments comprising a buttress end, engagement post, and an apex, wherein the engagement post connects the buttress end to the apex such that when pressure is applied to the buttress end the apex is impelled through the casting material; and
(c) one or more accoutrements that cooperatively interact with the one or more fitments to form an adorned orthopaedic cast.

9. The system of claim 8, further comprising an engagement post shell to facilitate application of the one or more fitments, wherein the engagement post shell reversibly envelopes the apex and the engagement post, and reversibly contacts an upper surface of the buttress end.

10. The system of claim 8, wherein the engagement post connects the buttress end and the apex, and wherein the engagement post is solid or has a hollow interior region.

11. The system of claim 8, wherein the apex extends beyond the plane of an exterior surface of the orthopaedic cast, or wherein the apex is excised.

12. The system of claim 8, wherein the one or more accoutrements cooperatively interact with the apex of the one or more fitments when the one or more accoutrements are substantially aligned with the apex.

13. The system of claim 8, wherein the one or more accoutrements cooperatively interact with the engagement post of the one or more fitments when the one or more accoutrements possess an engagement rod.

14. A method of adorning an orthopaedic cast comprising:
(a) providing a casting composition comprising at least one layer of reticulated material configured to conform to a body surface that defines a casting region;
(b) providing one or more fitments comprising a buttress end, engagement post, and an apex;
(c) perforating the casting composition with the one or more fitments such that the one or more fitments traverse the casting composition while the engagement post remains at least partially embedded;
(d) contacting the one or more fitments with one or more accoutrements capable of cooperatively interacting with the one or more fitments; and
(e) forming an adorned orthopaedic cast.

15. The method of claim 14 further comprising securing the one or more accoutrements to the apex or the engagement post of the one or more fitments such that the one or more accoutrements and the apex or the engagement post reversibly interact when the one or more accoutrements.

16. The method of claim 14 further comprising, excising the apex after the perforating, wherein the excising exposes an interior hollow region of the engagement post.

17. The method of claim 14 further comprising providing an engagement post shell to facilitate the perforating.

18. The method of claim 17, wherein the engagement post shell reversibly envelopes the apex and the engagement post, and wherein the engagement post shell reversibly contacts an upper surface of the buttress end.

19. The method of claim 14, wherein the apex extends beyond the plane of an exterior surface of the cast, or wherein the apex is excised and an exposed region of the engagement post is flush with an exterior surface of the cast.

20. The method of claim 14, wherein the engagement post has a hollow interior region, and wherein the engagement post is configured to secure the one or more fitments in place, and further wherein the one or more accoutrements comprise an engagement rod that interacts with the hollow interior region.
